(19) United States
(12) Patent Application Publication
(10) Pub. No.: US 2005/0036995 A1
(43) Pub. Date: Feb. 17, 2005

(54) Genetic vaccines for cancer therapy

(76) Inventors: Hans Herweijer, Madison, WI (US); Mary Kay Bates, Middleton, WI (US); Vladimir G. Budker, Middleton, WI (US); James E. Hagstrom, Middleton, WI (US); Sean D. Monahan, Madison, WI (US); David B. Rozema, Madison, WI (US); Paul M. Slattum, Madison, WI (US); John A. Wolff, Madison, WI (US)

Correspondence Address: MIRUS CORPORATION, 505 SOUTH ROSA RD, MADISON, WI, 53719, US

(21) Appl. No.: 10/892,882

(22) Filed: Jul. 16, 2004

Related U.S. Application Data
(63) Continuation-in-part of application No. 09/992,957, filed on Nov. 13, 2001.
(63) Continuation-in-part of application No. 10/600,098, filed on Jun. 20, 2003.
Application No. 10/600,098 is a division of application No. 09/447,966, filed on Nov. 23, 1999, now Pat. No. 6,627,616.
Application No. 09/447,966 is a continuation-in-part of application No. 09/391,260, filed on Sep. 7, 1999, now abandoned.
Application No. 09/391,260 is a division of application No. 08/975,573, filed on Nov. 21, 1997, now Pat. No. 6,265,387.
Application No. 08/975,573 is a continuation of application No. 08/571,536, filed on Dec. 13, 1995, now abandoned.
(60) Provisional application No. 60/488,478, filed on Jul. 18, 2003.
(60) Provisional application No. 60/248,275, filed on Nov. 14, 2000.

(51) Int. Cl. A61K 48/00; C12N 15/85

(52) U.S. Cl. 424/93.21; 435/455

(57) ABSTRACT

The present invention relates to methods for delivering a genetic immunogen comprising a polynucleotide capable of expressing an antigen. The polynucleotide is delivered to the host via an intravascular route resulting in delivery to extravascular cells, expression of an encoded antigen and induction of an antigen-specific immune response. The methods may be used to enhance an immune response against a cancer cell related antigen.

### CROSS-REFERENCE TO RELATED APPLICATIONS

This application claims the benefit of U.S. Provisional Application No. 60/488,478, filed Jul. 18, 2003, and is a continuation-in-part of application Ser. No. 09/992,957, filed Nov. 14, 2001, and a continuation-in-part of application Ser. No. 10/600,098 filed Jun. 20, 2003, which is divisional of application Ser. No. 09/447,966 filed Nov. 23, 1999, now U.S. Pat. No. 6,627,616, which is a continuation-in-part of application Ser. No. 09/391,260, filed on Sep. 7, 1999, which is a divisional of application Ser. No. 08/975,573, filed no Nov. 21, 1997, now U.S. Pat. No. 6,267,387, which is a continuation of application Ser. No. 08/571,536, filed on Dec. 13, 1995, now abandoned. Application Ser. No. 09/992,957 claims the benefit of U.S. Provisional Application No. 60/248,275, filed Nov. 14, 2000. U.S. Pat. No. 6,627,616 and U.S. application Ser. No. 09/992,957 are incorporated herein by reference.

### BACKGROUND OF THE INVENTION

Vaccination is the process of preparing an animal to respond to an antigen. Typical vaccination schemes produce a humoral immune response. They may also provide cytotoxic immunity. The humoral system protects a vaccinated individual from subsequent challenge from a pathogen and can prevent the spread of an intracellular infection if the pathogen goes through an extracellular phase during its life cycle; however, it can do relatively little to eliminate intracellular pathogens. Cytotoxic immunity complements the humoral system by eliminating the infected host cells. The most effective vaccinations activate both types of immunity.

The immune system of vertebrates consists of several interacting components. Two of the most important components are the humoral and cellular (cytolytic) branches. Antibody molecules, the effectors of humoral immunity, are secreted by special B lymphoid cells, B cells, in response to antigen. Antibodies can bind to and inactivate antigen directly (neutralizing antibodies) or activate other cells of the immune system to destroy the antigen. Cellular immune recognition is mediated by a special class of lymphoid cells, the cytotoxic T cells or cytotoxic T lymphocytes (CTLs). These cells respond to peptide fragments which appear on the surface of a target cell bound to major histocompatibility complex (MHC) proteins. The cellular immune system is constantly monitoring the proteins produced in all cells in the body in order to eliminate any cells producing foreign antigens. Humoral immunity is mainly directed at antigens which are exogenous to the animal whereas the cellular system responds to antigens which are actively synthesized within the animal.

Cells of the immune system include antigen presenting cells, which process antigens and present them to other immune cells to stimulate one of the two pathways, helper T cells, T-effector lymphocytes, natural killer cells, polymorphonuclear leukocytes, macrophages, dendritic cells, basophils, neutrophils, eosinophils, monocytes.

The development of vaccines is frequently heralded as one of the most important medical breakthroughs. Prevention of disease has increased human life expectancy, lowered healthcare costs, and enhanced quality of life. Yet more widespread use is hampered by the difficulty in creating effective vaccines for new microbes and the expense associated with distribution and administration of current vaccines. Gene transfer can also be used as a vaccination and can address the problems associated with conventional vaccines.

When a foreign gene is transferred to a cell and expressed, the resultant protein is presented to the immune system. With a classic vaccine, the antigen itself is introduced into the host—either in the form of attenuated, killed or inactivated microbe, or as purified (usually recombinant) protein, or as a synthesized peptide. With a genetic vaccine, the coding sequence for the antigen (or part of the antigen) is introduced into the host. Following transfection of the coding sequence into a host cell, the antigen is produced in situ. This presentation differs from the antigen presentation resulting from simply injecting the protein into the body and is more likely to cause a cell-mediated immune response. Expression of the antigen on the surface of a cell in the context of the major histocompatibility complex (MHC) is expected to result in a more appropriate, vigorous and realistic immune response, such as is frequently observed with attenuated virus vaccines. Also, no protein purification, or infectious agent preparation is necessary. With genetic immunization, truncations or added domains can be created by modification of the encoding polynucleotide. Also with genetic immunization, expression of a viral gene within a cell simulates a viral infection without the danger of an actual viral infection and induces a more effective immune response. This approach may be more effective in fighting latent viral infections such as human immunodeficiency virus, Herpes Simplex virus and cytomegalovirus.

Current genetic vaccination/immunization uses one of three methods: (1) direct injection of polynucleotide, such as naked DNA, into tissue such as skeletal muscle (optionally followed by electroporation); (2) ballistic delivery of plasmid DNA into the epidermis: gene gun (Chambers R S et al 2003); and (3) oral delivery of plasmid DNA (pDNA) formulations. Genetic vaccines have proven effective in eliciting immune responses against a wide variety of microbes. Protection in animal models has been demonstrated for influenza virus, malaria, bovine herpes virus, rabies virus, papilloma virus, herpes simplex virus, mycoplasma, lymphocytic choriomeningitis and others. The art has established that direct injection of pDNA into muscle is an efficient, reliable method for genetic vaccine delivery. However, gene transfer following intramuscular injection of pDNA is less efficient in larger rodents and primates. The genetic vaccine trials have corroborated these earlier gene transfer and expression studies, by finding the need to inject large amounts of pDNA in human muscles to obtain good immune responses. Complexing pDNA with cationic liposomes (lipoplexes) has been attempted to enhance the efficiency of intramuscular and intranasal delivery.

Immune responses following genetic vaccination/immunization have been reviewed in detail (Donnelly J J et al. 1997; Pardoll D M et al. 1995). Genetic vaccinations result in the induction of strong cytotoxic T lymphocyte (CTL) responses, where conventional subunit vaccines are skewed toward humoral responses. Since each individual genetic vaccine requires just the coding sequence for the antigen, many different vaccines can be produced and tested for each microbe. It is even feasible to generate a shot-gun library for a given microbe, vaccinate an appropriate animal model, and determine which clones result in the greatest immunity (either humoral or cellular). Alternatively, the expression of multiple epitopes allows genetic vaccines to better cover the variability in antigen presentation that exists in the population due to major histocompatibility (MHC) polymorphism. Because antigen expression has the potential to be maintained over a period of time, single dose immunization may also be possible with genetic immunization.

Genetic vaccines elicit both strong humoral and T cell responses, thus providing better memory activity against microbes such as malaria. The effectiveness of DNA vaccines to produce both humoral and cellular immunity indicates that DNA is expressed after administration, with the protein or peptide product being presented as an antigen in association with either Class I or Class II proteins. Myofibers can present antigen on MHC-I molecules, but appear to lack the co-stimulatory signals required for productive responses.

Antigen leaked from myofibers may be taken up by APCs (e.g., in the draining lymph nodes) that can subsequently provide strong stimulation (cross-priming). The immune response can be tailored by co-expression of cytokines. For instance expression of IL-12 or interferon-γ skews the response toward Th1 whereas co-expression of IL-4 results in a Th2 type response. Th1 Helper T Cells are essential for controlling such intracellular pathogens as viruses and certain bacteria, e.g., Listeria and Mycobacterium tuberculosis (the bacillus that causes tuberculosis). Th2 Helper T Cells provide help for B cells and, in so doing, are essential for antibody-mediated immunity. Antibodies are needed to control extracellular pathogens (which—unlike intracellular parasites—are exposed to antibodies in blood and other body fluids). Many publications have recently shown the effects of co-expression of interleukins and other cytokines, which should allow for fine tuning of the immune response following administration of genetic vaccines. Alternatively, it has been suggested that small numbers of professional Antigen Presenting Cells (APCs) are directly transfected and are responsible for the induction of the complete immune response. It has been hypothesized that transfer of antigen from myogenic cells to professional APCs can occur, thus obviating a requirement for direct transfection of bone marrow-derived cells (such as B-cells, T-cells, and APCs).

Delivery of nucleic acid expression vectors to suitable immune cells at one or more time points will allow for efficient generation of an antibody response. This immune response can immunize an animal against a concurrent or subsequent injection. Antibodies can also be subsequently obtained from the immunized host (e.g., production of polyclonal antibodies by bleeding). Alternatively, monoclonal antibody-producing hybridoma cells can be made by fusing antibody producing B (plasma) cells from the immunized host (e.g., spleen cells) with myeloma cells. Alternatively, the plasma cells can be immortalized, e.g., by retroviral transduction of ABL-Myc. Antibodies can be obtained from immortalized plasma cells (ascites) or hybridoma cells following culture in vitro or in vivo. Alternatively, T cell clones can be generated. Genetic immunization is extremely attractive for those investigators who have difficulty purifying a given protein or synthesizing a peptide. Also, those who already have cDNAs in mammalian expressions vectors can make antibodies quickly.

### SUMMARY OF THE INVENTION

The present invention provides methods for delivering an antigen to a vertebrate in vivo comprising: introducing a polynucleotide coding for the antigen into a vessel in the vertebrate whereby the polynucleotide is delivered into the interior of a cell in the vertebrate and the antigen is expressed and presented to the immune system of the vertebrate. The polynucleotide may code for an immunogenic peptide that is expressed by the transfected cells thereby generating an antigen-specific immune response. Generation of the immune response immunizes the vertebrate. Generation of the immune response also provides a method producing polyclonal antibodies, monoclonal antibodies, or immune cells of interest.

The methods can be used for the production of antibodies in a mammal, to provide a vaccine, or to provide a therapeutic response, such as to cancer or infection. In a preferred embodiment, methods are described for vaccinating, or immunizing, a vertebrate, comprising: forming an expressible polynucleotide encoding an antigen; and, injecting the polynucleotide into a vessel in the vertebrate thus delivering the polynucleotide to a cell in the vertebrate wherein the translation product of the polynucleotide, the antigen, is formed by the cell thereby eliciting an immune response against the antigen. The polynucleotide is injected into the vessel using a volume and rate sufficient to elevate intravascular pressure and increase permeability of tissue vasculature to the polynucleotide. The antigen may be delivered to a variety of cell types using the methods of the present invention, including, but not limited to, extravascular cells, liver cells, spleen cells, heart cells, lymph node cells, skeletal muscle cells, lung cells, thymus cells, kidney cells, skin cells, pancreas cells, intestinal cells, mucosal cells, antigen presenting cells, T cells, B cells, and macrophages. The antigen may be secreted by the cells, or it may be presented by a cell of the vertebrate in the context of a major histocompatibility antigen. The method may be used to selectively elicit a humoral immune response, a cellular immune response, or a mixture of these. In a preferred embodiment the antigen-encoding polynucleotide is introduced into the tail vein of a rodent. In another preferred embodiment, the polynucleotide is injected into a blood vessel in a vertebrate. In another preferred embodiment, the polynucleotide is injected into a limb vein of the vertebrate.

In an additional preferred embodiment, the antigen-encoding nucleic acid is rapidly introduced into the tail vein of a rodent in a relatively large volume of a pharmaceutically acceptable carrier, resulting a transiently elevated intravascular pressure.

In a preferred embodiment the polynucleotide may be introduced into the vertebrate using an injectable carrier alone. The carrier preferably is isotonic, hypotonic, or weakly hypertonic, such as provided by a sucrose, saline, or Ringer's solution. The polynucleotide may also be associated with or complexed with other compounds prior to injection of the polynucleotide into the vertebrate.

In a preferred embodiment the transferred polynucleotide expresses an antigen that induces an antigen-specific immune response. The antigen-specific immune response results in the formation of antigen-specific antibodies. The antigen-specific antibodies may be obtained and purified from the blood of the host. In a preferred embodiment B cells that produce antigen-specific antibodies may be obtained from the host. The B cells may be fused with myeloma cells to create monoclonal antibody producing cells. In another preferred embodiment the genetic immunization results in the induction of an antigen-specific cellular immune response. The immune response may result in the induction of T cells and/or natural killer (NK) cells.

In a preferred embodiment, the polynucleotide encodes an antigen of an intracellular infectious agent or an antigen encoded by a cellular gene. An intracellular infectious agent may be a viral pathogen, a bacterial pathogen, a fungal pathogen, a protozoan, or other intracellular pathogen. A cellular gene may a gene that is expressed in a cancer or tumor cell. The antigen is expressed in a cell and presented in the context of the MHC complex thereby stimulating a cellular immune response. The immune response may stimulate cytotoxic T cells that are capable of destroying infected or cancer/tumor cells. In another preferred embodiment, the polynucleotide encodes an extracellular antigen. The antigen may be expressed from the polynucleotide inside the cell and secreted by the cell.

In a preferred embodiment, the polynucleotide may be co-delivered with another agent to modulate or induce an immune reaction. The agent may be a polynucleotide, drug, protein, or other compound known to enhance, alter, augment, or inhibit one or more types of immune response.

In a preferred embodiment, polynucleotides may be delivered to extravascular limb cells to provide for expression of a peptide or protein antigen. We show that intravenous administration of a polynucleotide-containing solution results in delivery of the polynucleotide to nonvascular parenchymal cells, including skeletal muscle cells, expression of a gene encoded by the polynucleotide in the cells, and induction of an immune response in the mammal. The polynucleotide can encode a peptide or protein antigen to generate an immune response in the animal. The described process can be used for the production of antibodies in a mammal, to provide a vaccine, or to provide a therapeutic response, such as to cancer or infection.

Further objects, features, and advantages of the invention will be apparent from the following detailed description when taken in conjunction with the accompanying drawings.

### BRIEF DESCRIPTION OF THE FIGURES

FIG. 1. Graph illustrating reduced tumor growth in animals genetically immunized with a expression vector encoding a tumor antigen. Lymph node=direct injection of polynucleotide into lymph node. Spleen=direct injection of polynucleotide into spleen. Tail vein=injection of polynucleotide intravascularly into tail vein. Control=unimmunized mice.

FIG. 2. Anti-luciferase antibody titers in mice genetically immunized via delivery by hydrodynamic tail vein injection, direct intramuscular injection, and intravascular DNA/PEI/PAA particle injection.

FIG. 3. Immunohistochemical staining of ICR mouse skeletal muscle with antisera from mice genetically immunized with a polynucleotide encoding human dystrophin. The left panel shows muscle stained with the anti-human dystrophin antisera using a labeled anti-mouse IgG secondary antibody for fluorescence detection. The right panel shows staining with human-specific anti-human dystrophin monoclonal antibody.

FIG. 4. Western blots illustrating presence of antibodies to mammalian proteins in mice immunized with polynucleotides encoding human CD4 or canine dystrophin. The left panel shows detection of antigen using antisera from mice injected with CD4 encoding polynucleotide (predicted size 46 kD). The right panel shows detection of antigen using antisera from mice injected with dystrophin encoding polynucleotide (predicted size 425 kD). Each set of two lanes (− and +) represents serum from an individual mouse (− lane=cell extract lacking antigen; + lane=cell extract containing antigen).

FIG. 5. Immunohistochemical staining of HeLa cells probed with monoclonal antibody sera generated via intravascular genetic immunization of mice. Panel A shows Transduction Laboratories control anti-Ki67 monoclonal antibody (used at 1 μg/ml) generated via classical protein purification and injection. Panels B-F show five different culture supernatants from hybridoma fusions generated from mice immunized against Ki67 using intravascular delivery of polynucleotide. Secondary antibody was Cy3-labeled anti-mouse IgG (H+L) F(ab′)2 fragment.

FIG. 6. Western blot showing induction of luciferase-specific antibodies in rats following intravascular genetic immunization. The blot contains cell extracts for COS7 cells either expressing a control protein (− lanes) or luciferase (+ lanes). Rat antisera were used at a 1:100 dilution. Secondary anti-rat HRP antibody (Sigma) was used at a 1:5000 dilution.

FIG. 7. Antibody production against luciferase protein by genetic immunization of rabbits limb vein injection of antigen expressing polynucleotides. The left panel shows time course of antibody expression detected via ELISA. The right panel shows a Western blot using serum from immunized rabbit. The blot contained cell extracts for COS7 cells either expressing a control protein (− lanes) or luciferase (+ lanes). Rat antisera were used at a 1:100 dilution. Secondary anti-rat HRP antibody (Sigma) was used at a 1:5000 dilution.

FIG. 8. Graph illustrating immune response in mice immunized with different expression vectors with or without booster injection. Legend indicates expression vector used to drive luciferase expression. CMV=cytomegalovirus promoter vector. UbC=ubiquitin C promoter vector. The mice were immunized with plasmid DNA vectors expressing luciferase under transcriptional control of the CMV or the ubiquitin C promoter.

FIG. 9. Duplicate transfected HeLa cell lysates were run in two SDS polyacrylamide gels and each gel was transferred to Hybond-P (Amersham Biosciences). One blot was probed with chicken anti-NS1 IgY and the other was probed with rabbit anti-NS2 serum. Blots were developed using appropriately conjugated secondary antibodies and chemiluminescent detection.

### DETAILED DESCRIPTION OF THE INVENTION

We describe methods to elicit an antigen-specific immune response in a vertebrate via genetic immunization. Genetic immunization comprises delivering to a cell in vivo a polynucleotide encoding one or more antigens against which an immune response is to be generated. For genetic immunization to generate an antigen-specific immune response, the gene of interest must be delivered to host cells and expressed. The described methods comprise delivery systems for polynucleotides in vivo. The in vivo delivery and expression of the polynucleotide results in an immune response directed against an encoded antigen. A polynucleotide encoding an antigen (immunogen or immunogenic polypeptide) of interest is injected into a vessel of a vertebrate in a volume and at a rate that facilitate increasing permeability of vasculature in the vertebrate and delivery of the polynucleotide to an extravascular cell. The delivered polynucleotide is then expressed, producing the antigen in vivo.

The immune response may result in the formation of antigen-specific antibodies, the induction of an antigen-specific cellular immune response, the induction of an antigen-specific T cell response or the induction of natural killer cells. The immune response may directed against proteins associated with conditions, infections, diseases or disorders such as pathogen antigens or antigens associated with cancer cells.

The polynucleotide may be delivered to a cell in vivo to elicit a cell mediated immune response. The polynucleotide may also be delivered to a cell in vivo to elicit a humoral response. Cell mediated immunity is mediated by cells or the products they produce, such as cytokines, rather than by antibody production. It includes, but is not limited to, delayed type hypersensitivity and cytotoxic T cells. The term humoral immunity relates to an immune response mediated by antibodies and the cells involved in the production of antibodies. Cell mediated and humoral immunity are often induced simultaneously and influence each other. Since the immune systems of all vertebrates operate similarly, the applications described can be implemented in all vertebrate systems, comprising mammalian and avian species, as well as fish.

For vaccination purposes, the genetic vaccine is injected into a vessel in a vertebrate and delivered to cells of the vertebrate. The coding sequence of the expression cassette is expressed and the immunogenic polypeptide is produced. An immune response is then induced by the vertebrate against the immunogenic polypeptide. The immune response can be directed against proteins associated with conditions, infections, diseases or disorders such as allergens, pathogen antigens, antigens associated with cancer cells or cells involved in autoimmune diseases. The vaccinated individual may be immunized prophylactically or therapeutically to prevent or treat conditions, infections, diseases or disorders. The immunogenic polypeptide refers to peptides or proteins encoded by gene constructs of the present invention which act as target proteins for an immune response. The immunogenic protein shares at least an epitope with a protein from the allergen, pathogen, protein or cell-type such as an infected cell, a cancer cell or a cell involved in autoimmune disease against which immunization is desired. The immune response directed against the immunogenic polypeptide can protect the individual against and treat the individual for the specific infection or disease with which the polypeptide from the allergen, pathogen or undesirable protein or cell-type is associated. The immunogen does not need to be identical to the protein against which an immune response is desired. Rather, the immunogenic target polypeptide must be capable of inducing an immune response that cross reacts with the protein against which the immune response is desired.

Genetic immunization may be used to provide a method to treat latent viral infections. Several viruses, such as Hepatitis B, HIV and Herpes viruses, can establish latent infections in which the virus is maintained intracellularly in an inactive or partially active form. By inducing a cellular immune response against such viral infections, the infected cells can be targeted and eliminated. Chronic pathogen infections or poorly immunogenic infections may be similarly treated. There are numerous examples of pathogens which replicate slowly and spread directly from cell to cell. CTL directed killing of the infected cells can eliminate or slow the disease. The genetic immunization can be used to generate an immune response against infectious pathogens selected from the list comprising: immunodeficiency virus, human hepatitis A virus, human hepatitis B virus, human hepatitis C virus, influenza virus, smallpox (variola) virus, human herpes virus (type I through VIII), Bacillus, Bordetella, Borrelia, Brucella, Chlamydia, Clostridium, Corynebacterium, Escherichia, Haemophilus, Legionella, Listeria, Mycobacterium, Mycoplasma, Neisseria, Rickettsia, Salmonella, Staphylococcus, Streptococcus, Treponema, Vibrio, Yersinia, fungal pathogens, and pathogenic protozoans.

Genetic immunization can also be used to treat established diseases, such as but not limited to: cancer, tumor, and autoimmune disease. A number of tumor antigens which are recognized by T lymphocytes of the immune system have been identified and are considered as potential vaccine candidates. Therapeutic vaccination to mount a cellular immune response to a protein specific to the malignant state, be it an activated oncogene, a fetal antigen or an activation marker, may result in the elimination of these cells.

The immune response may be aimed at obtaining antibodies or immune cells specific for the antigen, for example B cells producing antibodies. These immune cells or immune cell products may be used for analytical or therapeutic purposes. As demonstrated by the data herein, the genetic immunization methods of the present invention provides substantially higher immune response efficiencies than available systems. Genetically immunized animals may be used to produce monoclonal antibodies. The means for preparing and characterizing antibodies are well known in the art.

Any peptide-based antigen which is a candidate for an immune response, whether humoral or cellular, can be used in its polynucleotide form. The genetic immunization may comprise a single injection of polynucleotide, a prime injection. Alternatively, the genetic immunization may comprise multiple injections of the polynucleotide, an initial prime injection and one or more subsequent boost (or booster) injections. Boosting can be repeated until a suitable titer or desired level of immune response is achieved.

The described immunization system comprises an intravascular administration route for the polynucleotide. Vessels comprise internal hollow tubular structures connected to a tissue or organ within the body of an animal, including a mammal. Bodily fluid flows to or from the body part within the lumen of the tubular structure. Examples of bodily fluid include blood, lymphatic fluid, or bile. Vessels comprise: arteries, arterioles, capillaries, venules, sinusoids, veins, lymphatics, and bile ducts. Afferent vessels are directed towards the organ or tissue and in which fluid flows towards the organ or tissue under normal physiological conditions. Conversely, efferent vessels are directed away from the organ or tissue and in which fluid flows away from the organ or tissue under normal physiological conditions. In the liver, the hepatic vein is an efferent blood vessel since it normally carries blood away from the liver into the inferior vena cava. Also in the liver, the portal vein and hepatic arteries are afferent blood vessels in relation to the liver since they normally carry blood towards the liver. A vascular network consists of the directly connecting vessels supplying and/or draining fluid in a target organ or tissue.

The choice of injection volume and rate are dependent upon: the size of the animal, the size of the vessel into with the solution is injected, the size and or volume of the target tissue, the bed volume of the target tissue vasculature, and the nature of the target tissue or vessels supplying the target tissue. For example, delivery to liver may require less volume because of the porous nature of the liver vasculature. The precise volume and rate of injection into a particular vessel, for delivery to a particular target tissue, may be determined empirically. Larger injection volumes and/or higher injection rates are typically required for a larger vessels, target sizes, etc. For example, efficient delivery to mouse liver may require injection of as little as 1 ml or less (animal weight ˜25 g). In comparison, efficient delivery to dog or nonhuman primate limb muscle may require as much as 60-500 ml or more (animal weight 3-14 kg). Injection rates can vary from 0.5 ml/sec or lower to 4 ml/sec or higher, depending on animal size, vessel size, etc. Occlusion of vessels, by balloon catheters, clamps, cuffs, natural occlusion, etc, can limit or define the vascular network size or target area.

Injecting into a vessel an appropriate volume at an appropriate rate increases permeability of the vessel to the injection solution and the molecules or complexes therein and increases the volume of extravascular fluid in the target tissue. Permeability can be further increased by injecting the polynucleotide while occluding outflow of fluid (both bodily fluid and injection solution) from the tissue or local vascular network. Permeability is defined herein as the propensity for macromolecules such as nucleic acids to move through vessel walls and enter the extravascular space. One measure of permeability is the rate at which macromolecules move through the vessel wall and out of the vessel. Another measure of permeability is the lack of force that resists the movement through the vessel wall and out of the vessel. Vessels contain elements that prevent macromolecules from leaving the intravascular space (internal cavity of the vessel). These elements include endothelial cells and connective material (e.g., collagen). Increased permeability indicates that there are fewer of these elements that can block the egress of macromolecules and/or that the spaces between these elements are larger and more numerous. In this context, increased permeability enables a higher percentage of macromolecules being delivered to leave the intravascular space, while low permeability indicates that a low percentage of the macromolecules will leave the intravascular space.

Vasculature permeability may be further increased by increasing the osmotic pressure within the vessel. Typically, hypertonic solutions containing salts such as sodium chloride, sugars or polyols such as mannitol are used. Hypertonic means that the osmolality of the injection solution is greater than physiologic osmolality. Isotonic means that the osmolality of the injection solution is the same as the physiological osmolality (i.e., the tonicity or osmotic pressure of the solution is similar to that of blood). Hypertonic solutions have increased tonicity and osmotic pressure compared to the osmotic pressure of blood and cause cells to shrink.

The permeability of the blood vessel can also be further increased by administering a biologically-active molecule such as a protein or a simple chemical such as histamine that increases the permeability of the vessel by causing a change in function, activity, or shape of cells within the vessel wall such as the endothelial or smooth muscle cells. Typically, biologically active molecules that affect permeability interact with a specific receptor or enzyme or protein within the vascular cell to change the vessel's permeability. Biologically active molecules include vascular permeability factor (VPF) which is also known as vascular endothelial growth factor (VEGF). Another type of biologically active molecule can also increase permeability by changing the extracellular connective material. For example, an enzyme could digest the extracellular material and increase the number and size of the holes of the connective material. Other biologically active molecules that may alter the permeability include calcium channel blockers (e.g., verapamil, nicardipine, diltiazem), beta-blockers (e.g., lisinopril), phorbol esters (e.g., PKC), ethylenediaminetetraacetic acid (EDTA), adenosine, papaverine, atropine, and nifedipine.

For genetic immunization, the transferred polynucleotide encodes polypeptide which is expressed and induces a desired immune response. The expressed antigen may be secreted by the cell or be presented by the cell in the context of the major histocompatibility antigens, thereby eliciting an immune response. The cell may be a professional antigen presenting cell or a non-profession antigen presenting cell. The antigen may be expressed in a non-APC and then taken up by an APC, in a process termed cross-priming (Larrson M et al. 2001; Clotilde T et al. 2001; Doe B et al. 1996). For example, the expressed antigen may leak from the transfected cell and be taken up by an APC (e.g., in the draining lymph nodes). The antigen may be released in the context of SOS signals, heat shock proteins, etc., and taken up by an APC. The APC then presents the antigen to other immune cells. The method may be used to selectively elicit a humoral immune response (B cell mediated), a cellular immune response (T-cell mediated), or a mixture of these.

An antigen refers to any agent that is recognized by an antibody. The term immunogen refers to any agent that can elicit an immunological response in an animal. In many cases, antigens are also immunogens, thus the term antigen is often used interchangeably with the term immunogen. These terms may be used to refer to an individual macromolecule or to a homogeneous or heterogeneous population of antigenic molecules. The antigenic moiety can also be a subunit of a protein, peptide, chimeric polypeptide, recombinant polypeptide or similar product. A chimeric polypeptide comprises two or more peptide sequences derived from different genes but expressed as a single polypeptide sequence. For genetic immunization, the antigen or immunogen is a polypeptide expressed from a delivered polynucleotide. The genetic immunization may e licit an immune response against a single antigen, or against a plurality of antigens.

Immunogenic peptide or immunogen is meant to refer to an antigen that is a target for an immune response and against which an immune response can be elicited. The immunogenic protein shares at least an epitope with a protein against which immunization is desired. In one application, the immune response is directed at proteins associated with conditions, infections, diseases or disorders such as allergens, pathogen antigens, antigens associated with cancer cells or cells involved in autoimmune diseases. In another application, the antigen-directed immune response is applied to (basic) biological studies, the generation of cellular or humoral immune response products (e.g., CTL clones, B cells, plasma cells, antibodies), or derivatives thereof (e.g., monoclonal antibodies). The immunogenic antigen is encoded by the coding sequence of a genetic construct called an expression vector.

The term antibody encompasses whole immunoglobulin of any class, chimeric antibodies, hybrid antibodies with dual or multiple antigen specificities and fragments including hybrid fragments. Also included within the meaning of antibody are conjugates of such fragments, and so-called antigen binding proteins (single chain antibodies) as described, for example, in U.S. Pat. No. 4,704,692. Alternatively, the encoded antibodies can be anti-idiotypic antibodies (antibodies that bind other antibodies) as described, for example, in U.S. Pat. No. 4,699,880.

The term polynucleotide, or nucleic acid or polynucleic acid, is a term of art that refers to a polymer containing at least two nucleotides. Nucleotides are the monomeric units of polynucleotide polymers. Polynucleotides with less than 120 monomeric units are often called oligonucleotides. Natural nucleic acids have a deoxyribose- or ribose-phosphate backbone. An artificial or synthetic polynucleotide is any polynucleotide that is polymerized in vitro or in a cell free system and contains the same or similar bases but may contain a backbone of a type other than the natural ribose-phosphate backbone. These backbones include: PNAs (peptide nucleic acids), phosphorothioates, phosphorodiamidates, morpholinos, and other variants of the phosphate backbone of native nucleic acids. Bases include purines and pyrimidines, which further include the natural compounds adenine, thymine, guanine, cytosine, uracil, inosine, and natural analogs. Synthetic derivatives of purines and pyrimidines include, but are not limited to, modifications which place new reactive groups such as, but not limited to, amines, alcohols, thiols, carboxylates, and alkylhalides. The term base encompasses any of the known base analogs of DNA and RNA including, but not limited to, 4-acetylcytosine, 8-hydroxy-N-6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyl-uracil, dihydrouracil, inosine, N6-isopentenyladenine, 1-methyladenine, 1-methyl-pseudouracil, 1-methylguanine, 1-methylinosine, 2,2-dimethyl-guanine, 2-methyladenine, 2-methylguanine, 3-methyl-cytosine, 5-methylcytosine, N6-methyladenine, 7-methylguanine, 5-methylaminomethyluracil, 5-methoxy-amino-methyl-2-thiouracil, P-D-mannosylqueosine, 5′-methoxycarbonylmethyluracil, 5-methoxyuracil, 2-methylthio-N-6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine. The term polynucleotide includes deoxyribonucleic acid (DNA) and ribonucleic acid (RNA) and combinations of DNA, RNA and other natural and synthetic nucleotides.

DNA may be in form of cDNA, in vitro polymerized DNA, plasmid DNA, parts of a plasmid DNA, genetic material derived from a virus, linear DNA, vectors (P1, PAC, BAC, YAC, artificial chromosomes), expression cassettes, chimeric sequences, recombinant DNA, chromosomal DNA, an oligonucleotide, anti-sense DNA, or derivatives of these groups. RNA may be in the form of oligonucleotide RNA, tRNA (transfer RNA), snRNA (small nuclear RNA), rRNA (ribosomal RNA), mRNA (messenger RNA), in vitro polymerized RNA, recombinant RNA, chimeric sequences, anti-sense RNA, siRNA (small interfering RNA), ribozymes, or derivatives of these groups. An anti-sense polynucleotide is a polynucleotide that interferes with the function of DNA and/or RNA. Antisense polynucleotides include, but are not limited to: morpholinos, 2′-O-methyl polynucleotides, DNA, RNA and the like. SiRNA comprises a double stranded structure typically containing 15-50 base pairs and preferably 19-25 base pairs and having a nucleotide sequence identical or nearly identical to an expressed target gene or RNA within the cell. Interference may result in suppression of expression. The polynucleotide can be a sequence whose presence or expression in a cell alters the expression or function of cellular genes or RNA. In addition, DNA and RNA may be single, double, triple, or quadruple stranded. Double, triple, and quadruple stranded polynucleotide may contain both RNA and DNA or other combinations of natural and/or synthetic nucleic acids.

A polynucleotide can be delivered to a cell to express an exogenous nucleotide sequence, to inhibit, eliminate, augment, or alter expression of an endogenous nucleotide sequence, or to affect a specific physiological characteristic not naturally associated with the cell. Polynucleotides may contain an expression cassette coded to express a whole or partial protein, or RNA. An expression cassette refers to a natural or recombinantly produced polynucleotide that is capable of expressing a sequence. The term recombinant as used herein refers to a polynucleotide molecule that is comprised of segments of polynucleotide joined together by means of molecular biological techniques. The cassette contains the coding region of the gene of interest along with any other sequences that affect expression of the sequence of interest. An expression cassette typically includes a promoter (allowing transcription initiation), and a transcribed sequence. Optionally, the expression cassette may include, but is not limited to, transcriptional enhancers, non-coding sequences, splicing signals, transcription termination signals, and polyadenylation signals. An RNA expression cassette typically includes a translation initiation codon (allowing translation initiation), and a sequence encoding one or more proteins. Optionally, the expression cassette may include, but is not limited to, translation termination signals, a polyadenosine sequence, internal ribosome entry sites (IRES), and non-coding sequences. The regulatory sequences of the expression cassette may be selected to be appropriate for the target cell and host. The choice of regulatory sequences in the expression cassette may also depend on the duration of expression desired. For some applications, it is desirable that the antigen be expressed for a short period of time. For other applications, it may be longer term expression may be desired.

The polynucleotide may contain sequences that do not serve a specific function in the target cell but are used in the generation of the polynucleotide. Such sequences include, but are not limited to, sequences required for replication or selection of the polynucleotide in a host organism.

The term naked polynucleotide indicate that the polynucleotide is not associated with a transfection reagent or other delivery vehicle that is required for the nucleic acid or polynucleotide to be delivered to the cell. A transfection reagent is a compound or compounds that bind(s) to or complex(es) with oligonucleotides and polynucleotides, and mediates their entry into cells. The transfection reagent also mediates the binding and internalization of oligonucleotides and polynucleotides into cells. Examples of transfection reagents include, but are not limited to, cationic lipids and liposomes, polyamines, calcium phosphate precipitates, histone proteins, polyethylenimine, and polylysine complexes. It has been shown that cationic proteins like histones and protamines, or synthetic cationic polymers like polylysine, polyarginine, polyornithine, DEAE dextran, polybrene, and polyethylenimine may be effective intracellular delivery agents. Typically, the transfection reagent has a net positive charge that binds to the oligonucleotide's or polynucleotide's negative charge. The transfection reagent mediates binding of oligonucleotides and polynucleotides to cells via its positive charge (that binds to the cell membrane's negative charge) or via cell targeting signals that bind to receptors on or in the cell. For example, cationic liposomes or polylysine complexes have net positive charges that enable them to bind to DNA or RNA. Polyethylenimine, which facilitates gene transfer without additional treatments, probably disrupts endosomal function itself.

A non-viral vector is defined as a vector that is not assembled within an eukaryotic cell including protein and polymer complexes (polyplexes), lipids and liposomes (lipoplexes), combinations of polymers and lipids (lipopolyplexes), and multilayered and recharged particles.

The term gene generally refers to a polynucleotide sequence that comprises coding sequences necessary for the production of a therapeutic polynucleotide (e.g., ribozyme) or a polypeptide or precursor. The polypeptide can be encoded by a full length coding sequence or by any portion of the coding sequence so long as the desired activity or functional properties (e.g., enzymatic activity, ligand binding, signal transduction) of the full-length polypeptide or fragment are retained. The term also encompasses the coding region of a gene and the including sequences located adjacent to the coding region on both the 5′ and 3′ ends for a distance of about 1 kb or more on either end such that the gene corresponds to the length of the full-length mRNA. The sequences that are located 5′ of the coding region and which are present on the mRNA are referred to as 5′ untranslated sequences. The sequences that are located 3′ or downstream of the coding region and which are present on the mRNA are referred to as 3′ untranslated sequences. The term gene encompasses both cDNA and genomic forms of a gene. A genomic form or clone of a gene contains the coding region interrupted with non-coding sequences termed introns, intervening regions or intervening sequences. Introns are segments of a gene which are transcribed into nuclear RNA. Introns may contain regulatory elements such as enhancers. Introns are removed or spliced out from the nuclear or primary transcript; introns therefore are absent in the mature RNA transcript. The messenger RNA (mRNA) functions during translation to specify the sequence or order of amino acids in a nascent polypeptide. A gene may also includes other regions or sequences including, but not limited to, promoters, enhancers, transcription factor binding sites, polyadenylation signals, internal ribosome entry sites, silencers, insulating sequences, matrix attachment regions. These sequences may be present close to the coding region of the gene (within 10,000 nucleotides) or at distant sites (more than 10,000 nucleotides). These non-coding sequences influence the level or rate of transcription and/or translation of the gene. Covalent modification of a gene may influence the rate of transcription (e.g., methylation of genomic DNA), the stability of mRNA (e.g., length of the 3′ polyadenosine tail), rate of translation (e.g., 5′ cap), nucleic acid repair, nuclear transport, and immunogenicity. One example of covalent modification of nucleic acid involves the action of LabelIT reagents (Mirus Corporation, Madison, Wis.).

Condensing a polynucleotide means decreasing the volume that the polymer occupies. An example of condensing nucleic acid is the condensation of DNA that occurs in cells. The DNA from a human cell is approximately one meter in length but is condensed to fit in a cell nucleus that has a diameter of approximately 10 microns. The cells condense (or compact) DNA by a series of packaging mechanisms involving the histones and other chromosomal proteins to form nucleosomes and chromatin. The DNA within these structures is rendered partially resistant to nuclease DNase) action. The process of condensing polynucleotides can be used for delivering them into cells of an organism.

Two molecules are combined to form a complex—through a process called complexation or complex formation—if they are in contact with one another through noncovalent interactions such as electrostatic interactions, hydrogen bonding interactions, and hydrophobic interactions. An interpolyelectrolyte complex is a noncovalent interaction between polyelectrolytes of opposite charge.

Delivery of a polynucleotide means to transfer the polynucleotide from a container outside a vertebrate to near or within the outer cell membrane of a cell in the vertebrate. The term transfection is used herein, in general, as a substitute for the term delivery, or, more specifically, the transfer of a polynucleotide from directly outside a cell membrane to within the cell membrane. If the polynucleotide is a DNA or cDNA, it enters the nucleus where it is transcribed into a messenger RNA that is then transported into the cytoplasm where it is translated into a protein. If the nucleic acid is an mRNA transcript, it is translated in the cytoplasm by a ribosome to produce a protein. If the nucleic acid is an anti-sense nucleic acid it can interfere with DNA or RNA function in either the nucleus or cytoplasm.

A polyclonal antibody is prepared by immunizing an animal with an immunogenic composition in accordance with the present invention and collecting antisera from that immunized animal. A wide range of animal species can be used for the production of antisera. Antibodies may then be purified from the sera if desired. Typically the animal used for production of anti-antisera is selected from the group comprising: rabbit, mouse, rat, hamster, guinea pig, chicken, donkey, horse, and goat.

Genetically immunized animals may be used to produce monoclonal antibodies. The means for preparing and characterizing antibodies are well known in the art (See, e.g., Antibodies: A Laboratory Manual, Cold Spring Harbor Laboratory, 1988; incorporated herein by reference; and as those exemplified in U.S. Pat. No. 4,196,265, incorporated herein by reference). Typically, this technique involves immunizing a suitable animal with a selected immunogen composition, e.g., a purified or partially purified epitopic protein, polypeptide or peptide. The immunizing composition is administered in a manner effective to stimulate antibody producing cells. Following immunization, somatic cells with the potential for producing antibodies, specifically B lymphocytes (B cells), are selected for use in the mAb generating protocol. These cells may be obtained from spleens, tonsils or lymph nodes, or from a peripheral blood sample. Often, a panel of animals will have been immunized and spleen lymphocytes obtained the animal with the highest antibody titer. The antibody-producing B lymphocytes from the immunized animal are then immortalized (e.g., by retroviral transduction with ABL-Myc) or fused with cells of an immortal myeloma cell, generally one of the same species as the animal that was immunized. Any one of a number of myeloma cells may be used, as are known to those of skill in the art.

It is well known in the art that the immunogenicity of a particular immunogen composition can be enhanced by the use of non-antigen-specific stimulators of the immune response, known as adjuvants. An adjuvant is a compound that, when used in combination with an antigen, can augment or otherwise alter or modify the resultant immune responses. The present invention contemplates immunization with or without adjuvant. For immunization with an adjuvant, the invention is not limited to any particular type of adjuvant. Adjuvants may be used either separately or in combination. Adjuvants known in the art may be selected from the list comprising: complete Freund's adjuvant (a non-specific stimulator of the immune response containing killed Mycobacterium tuberculosis), incomplete Freund's adjuvants, a garbeads, aluminum hydroxide, aluminum phosphate (alum), Quil A adjuvant (commercially available from Accurate Chemical and Scientific Corporation), Gerbu adjuvant (commercially available from C.C. Biotech Corp.), and bacterin (i.e., killed preparations of bacterial cells).

Several means to enhance the immune response generated by genetic immunization are readily conceivable. For example, genes of compounds may be delivered to cells which increase the number of histocompatibility antigens on the cell surfaces. Polynucleotide delivery can also be combined with an agent to stimulate cytokine production or release, causing lymphocyte or other immune cell proliferation or activation. Interferons or interleukins, or polynucleotides expressing interferons or interleukins may also be delivered to the animal. The polynucleotide itself may also be covalently modified with a compound to enhance an immune response. Also, the polynucleotide associated with a ligand that directs it to a specific cell type.

Another method for increasing antibody induction is by formation of multimeric antigens, which can stimulate B cells without T help. This can be achieved by generating fusion of the antigen with pentraxin proteins (e.g., C reactive protein, serum amyloid protein) or IgM, which form pentamers. A similar approach was recently described to significantly enhance genetic immunization antibody induction using a cartilage oligomeric matrix protein sequence.

The immune response elicited by expressed antigens can be augmented or modulated by co-expression or administration of interleukins, cytokines, interferons, growth and differentiating factors, or specific cell surface-receptor ligands. These factors can promote humoral or cell-mediated response through mobilization, activation, repression, proliferation, or maturation of immune cells or effector cells, including T cells, Th1 helper T cells (which participate in cell-mediated immunity), Th2 helper T cells (which provide help for B cells), B cells, NK cells and professional antigen presenting cells such as dendritic cells. Factors such as IL-2 or IL-7 and Th1-biasing cytokines such as IFN-γ and IL-12 have been demonstrated to selectively enhance the induction of CTL-mediated immunity in mice. Alternatively, a diminished CTL responsiveness and an enhanced antigen-specific humoral response are observed with the co-delivery of Th2-biasing cytokines IL-4, IL-5, and IL-10 (Xiang Z et al. 1995; Chow Y H et al. 1998; Iwasaki A et al. 1997; Kim J J et al. 1997). Recent investigations have shown that DCs play a central role in the stimulation of cellular and humoral immunity following genetic immunization. Antigen can be endogenously expressed through direct gene-transfection of APCs, or can be acquired exogenously from transfection and expression by non-DC cell types (Tuting T et al. 1998). Genetic vaccine strategies involving co-delivery of Flt3-L or GM-CSF pDNA have shown significant increases in antigen-specific antibody generation and CTL-mediated protection (Sailaja G et al. 2003; Rakhmilevich A L et al. 2001; Sun X et al. 2002). CD154 (CD40 ligand), which promotes DC maturation, with genetic immunization has demonstrated both humoral and cellular antigen-specific immunological enhancement to antigens like HIV-1 encoded proteins (Ihata A et al. 1999). It is readily conceivable that prior treatment with certain stimulators will prime the host for a subsequent antigen delivery, and thus result in a stronger or more rapid antigen-specific immune response.

Combinations of immunomodulators may be used in accordance with the present invention. In addition, relative timing of administration of an immunomodulator may be important for maximal immune response or for eliciting the desired type of immune response.

### EXAMPLES

The following examples are provided in order to demonstrate and further illustrate certain preferred embodiments and aspects of the present invention and are not to be construed as limiting the scope thereof.

### Example 1

Reduction in tumor growth in mice following intravascular genetic immunization. C57B1/6 mice were immunized 4 times with 50 μg of a plasmid containing the human melanoma tumor antigen gp100 cDNA driven by the cytomegalovirus (CMV) promoter (pCI-hgp100). Mice were immunized via direct injection of the inguinal lymph node (group 1), direct intrasplenic injection (group 2), or tail vein injection under increased pressure (group 3). Five mice were immunized per group. Each animal was immunized on days 0, 14, 21 and 28.

For the lymph node delivery method, mice were shaved and prepped with an antiseptic solution. A small incision (1 to 2 cm) was made just above the groin and the skin was gently pried apart to expose the inguinal lymph node. The lymph node was directly injected with a 10-100 μl solution of DNA using a 0.5 ml or 1.0 ml syringe with a 30 gauge needle. The skin was then closed with 1 or 2 stitches using 4-0 Braunamid suture.

For the direct intrasplenic delivery method, mice were shaved and prepped with an antiseptic solution. A small incision (1 to 2 cm) was made on the left side of the abdomen just below the rib cage. The skin was gently pried apart and the peritoneum opened to expose the spleen. The spleen was directly injected with a 10-100 μl solution of DNA using a 0.5 ml syringe with a 30 gauge needle. The peritoneum and skin were closed with 1 or 2 stitches using 4-0 Braunamid suture.

For delivery via insertion into tail vein, 1.0 ml solution containing the plasmid DNA per 10 g animal body weight was inserted into the tail vein using a 30 gauge, 0.5 inch needle. Injections were done manually with injection times of 4-5 sec.

Mice were bled on days—1, 7, 20, 27 and 34. Blood was collected from the retro-orbital sinus. Serum was separated and stored for later analysis.

On day 38 mice were inoculated with 1×10⁵ syngeneic B16 melanoma cells stably transfected with the hgp100 antigen. Cells were cultured in RPMI 1640 (Cellgro) supplemented with 10% fetal bovine serum, 1% penicillin streptomycin and 2.5% HEPES pH 7.3. At 80% confluence, cells were harvested, counted and resuspended in 50 μl sterile PBS. Mice were shaved in the abdominal area and the cells were injected intradermally. Control mice were not immunized but received the same tumor inoculation as the immunized mice.

Tumors size was determined three times a week by measuring two perpendicular diameters using digital calipers. Tumor volume was calculated using the formula (length×width×width/2)=tumor volume. Mice were sacrificed when tumor diameter exceeded 1 cm³.

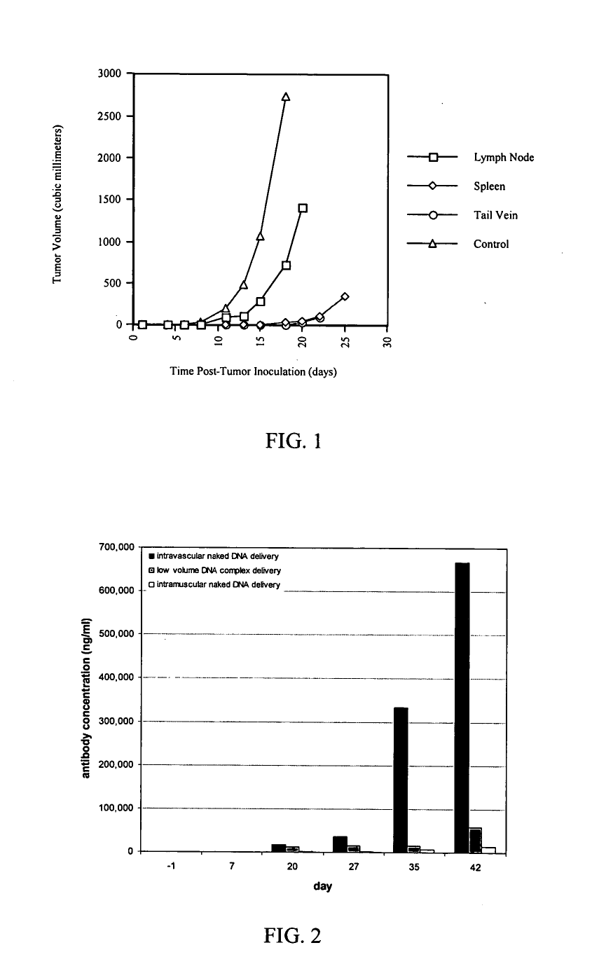

The results (FIG. 1) show that genetic immunization by delivery of DNA via tail vein injection is as effective in reducing model tumor growth in mice as direct splenic injection. 3 of 5 mice immunized via tail vein injection of polynucleotide did not develop tumors.

### Example 2

Comparison of alternate delivery routes for genetic immunization of mice. The luciferase expression vector pMIR48 was administered to ICR mice by each of three methods: intramuscular and intravascular delivery of naked pDNA, and intravascular delivery of pDNA particles (5 animal per group). For direct intramuscular injections in the quadriceps, 50 μg plasmid DNA in 100 μl saline was injected. For intravascular delivery via hydrodynamic tail vein injection, 50 μg plasmid DNA in 1 ml Ringer's solution per 10 g mouse body weight was injected in about 7 seconds. For low-pressure tail vein injection, 50 μg plasmid DNA was complexed with the polycation polyethylenimine (PEI) and recharged with the polyanion polyacrylic acid (PAA) at a ratio of 1:6:1 (wt:wt:wt) in a volume of 50 μl. Mice were injected on days 0, 14, 21 and 28. To quantitate anti-luciferase antibody titers, sequential serum samples were taken before the initial (prime) injection and 7 days after each injection and analyzed by standard ELISA test. A standard curve was generated using a commercially available anti-luciferase antibody. The results (FIG. 2) demonstrate that increased pressure intravascular delivery of naked pDNA resulted in higher titers and more rapid induction of anti-luciferase antibodies than the more conventional injection into skeletal muscle. Dose response experiments (not shown) have indicated that after two booster injections with 10 μg pDNA delivered IV resulted in higher titers than the highest dose delivered IM (100 μg). PEI/PAA particles are better than IM injection, even though the final titers are lower than after IV immunization.

### Example 3

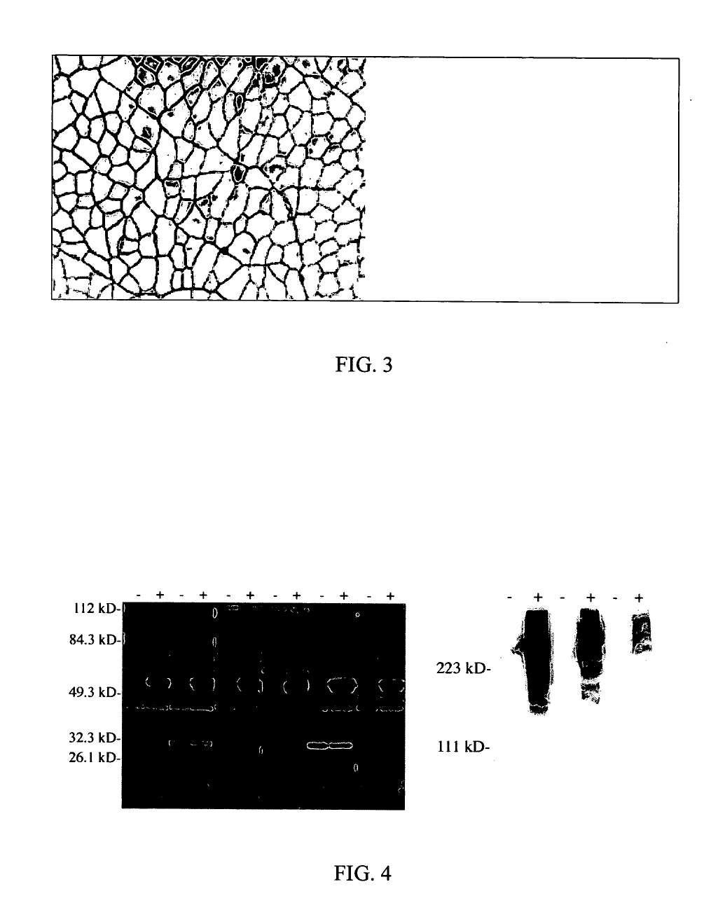

Generation of antibodies in mouse to human dystrophin. An anti-human dystrophin antibody was generated in ICR mice by genetic immunization. The mice were primed and boosted by high pressure tail vein delivery of 100 μg of a human dystrophin expression cassette (2 boosts at 2 and 3 weeks after the prime). Sera were obtained 3 days after the second boost and used to stain for human dystrophin expression in mdx (dystrophin deficient) mice previously injected with 10 μg of the same expression vector (IM). Immunohistochemistry with the antisera showed a the presence of myofibers expressing human dystrophin in a typical dystrophin staining pattern. These results were identical to those obtained with commercially available anti-human dystrophin antibodies. Thus intravascular genetic immunization can result in the generation of antibodies against clinically relevant target proteins with titers are sufficient to be used for immunohistochemistry. The antisera was further shown to cross react with the mouse dystrophin in ICR (dystrophin positive mice). Dystrophin staining in ICR mouse with the antisera is shown in FIG. 3. The left panel of FIG. 3 shows mouse skeletal muscle stained with the anti-human dystrophin polyclonal antisera using a labeled anti-mouse IgG secondary antibody for fluorescence detection. The right panel shows mouse skeletal muscle stained with a commercially available anti-human dystrophin monoclonal antibody that does not cross react with mouse dystrophin.

### Example 4

Comparison of intravascular genetic immunization to standard intramuscular injection. 50 or 100 μg of DNA encoding firefly luciferase were injected into mice 4 times as described above. The first injection, the prime injection, occurred at day 0. Subsequent injections occurred on days 14, 21 and 28. Antisera from mice were tested at various times before, during and after immunization. As shown in the table, genetic immunization by intravascular delivery of polynucleotide resulted in higher antigen-specific antibody titers than did intramuscular injection of polynucleotide. Similar results were observed in animals which received injections in which the DNA was in: a) standard Ringers' solution, b) standard Ringers' solution+5% mannitol or c) 50% standard Ringers' solution/50% saline+3.75% mannitol.

Levels of anti-luciferase antibody titers (μg/ml antibody concentration) generated by intravascular tail vein injection versus direct intramuscular injection. Levels shown are averages of five mice per group.

| | Intravascular | | Intramuscular | |
|---|---|---|---|---|
| day | 50 μg DNA | 100 μg DNA | 50 μg DNA | 100 μg DNA |
| 0 | 0.05 ± 0.03 | 0.04 ± 0.01 | 0.09 ± 0.05 | 0.04 ± 0.01 |
| 7 | 0.12 ± 0.04 | 0.23 ± 0.12 | 0.07 ± 0.02 | 0.06 ± 0.01 |
| 20 | 17.7 ± 20.3 | 20.3 ± 15.6 | 1.06 ± 1.70 | 1.85 ± 2.80 |
| 27 | 36.8 ± 25.9 | 79.2 ± 28.9 | 1.90 ± 2.42 | 5.98 ± 4.25 |
| 35 | 334 ± 244 | 344 ± 234 | 6.62 ± 9.57 | 3.73 ± 4.51 |
| 42 | 668 ± 366 | 926 ± 229 | 14.3 ± 21.8 | 17.4 ± 16.8 |

### Example 5

Generation of antibodies to mammalian antigens in mice. Mice were immunized, as described above for tail vein injection, with polynucleotides encoding either a truncated human CD 4 protein or canine dystrophin. CD4 represent a membrane-bound antigen and dystrophin represents an intracellular antigen. For both, mice were immunized by the intravascular tail vein procedure described above. 50 μg plasmid DNA injected into the tail vein on days 0, 14, 21 and 28. Blots, containing extracts from cells expressing either the immunizing antigen (+ lanes) or a control protein (− lanes) were probed with sera sampled on day 35. Sera were diluted 1:100. The CD4 protein has a predicted protein size of approximately 46 kD and the canine dystrophin has a predicted protein size of 425 kD. FIG. 4 shows that each of the mice produced antigen-specific antibodies.

### Example 6

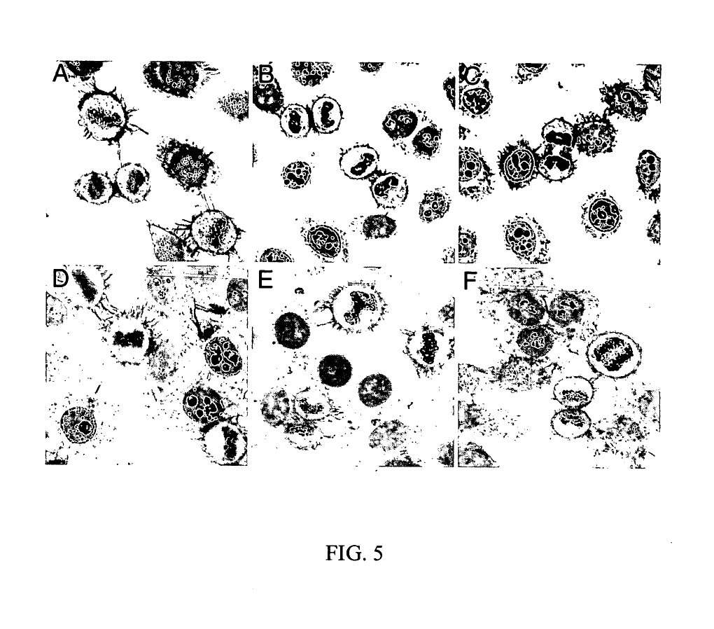

Hybridoma Fusion Using Splenocytes from Mice Immunized via Intravascular Delivery of a Plasmid. Six mice were immunized with pMIR167 encoding human Ki67, a chromatin-binding protein, via four injections into tail vein as described above. Analysis of the mouse antisera showed very strong signal (results not shown). Animal were given a fifth immunization and day 105 and spleens were harvested four days later. Splenocytes were frozen and processed for hybridoma fusion using methods standard in the art. 46 clones were isolated that presented typical Ki-67 pattern in immuno-cytochemical staining. None of the supernatants cross-reacted with mouse. Two cross-reacted with rat. Almost all cross-reacted with monkey Ki67. Five of these culture supernatants, along with a commercially available anti-Ki67 antibody are shown detecting Ki67 in HeLa cells in FIG. 5.

### Example 7

Antibodies generated via intravascular genetic immunization maintain high titers over long-term. Four mice were immunized via intravascular tail vein delivery of polynucleotides as described above. Mice were injected with 10 μg pMIR48 on days 0, 14, and 28. High titer was observed at day 48 as tested by ELISA, three weeks after the last boost. This level was maintained for at least another 32 days.

| | anti-luciferase antibody titer |
|---|---|
| day | (μg Ab/ml serum) |
| 0 | 0.01 |
| 13 | 0.02 |
| 20 | 0.39 |
| 27 | 5.40 |
| 34 | 8.76 |
| 41 | 16.5 |
| 48 | 46.9 |
| 76 | 48.5 |

### Example 8

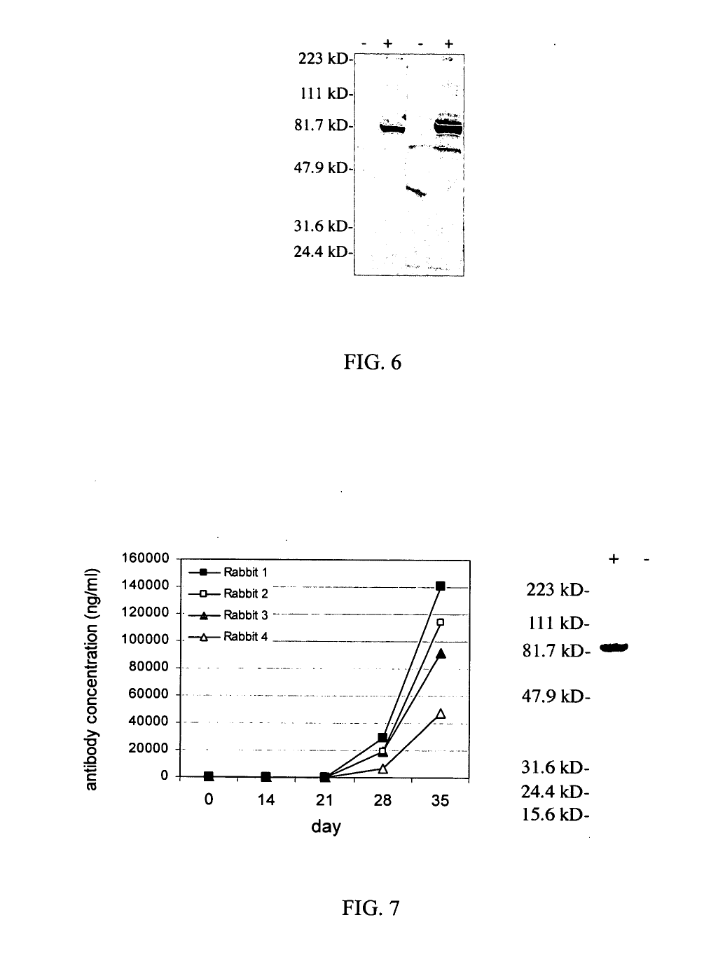

Intravascular genetic immunization in rats. Rats were genetically immunized via intravascular delivery of polynucleotide as described for mouse immunization. 500 μg pMIR48 in 20 ml was injected into the tail vein of rats in 20 sec. Rats were injected on days 0, 14, 21 and 28. On day 35 animals were bled and the sera were tested for the presence of anti-luciferase antibodies by Western blot. The data in FIG. 6 shows luciferase-specific antibodies were present in the injected rats (− lane=cell extract lacking antigen; + lane=cell extract containing antigen), demonstrating the application of the intravascular genetic immunization in larger rodents.

### Example 9

Induction of immune response in mice following intravenous delivery of a polynucleotide: Four mice were injected on days 0, 14 and 21 with a plasmid encoding the firefly luciferase gene under control of the cytomegalovirus promoter (pMIR48). For each injection, a solution containing the plasmid was inserted into lumen of the saphenous vein animals as described in U.S. application Ser. No. 10/855,175 (incorporated herein by reference) and as follows: A latex tourniquet was wrapped around the upper hind limb just above the quadriceps and tightened into place with a hemostat to block blood flow to and from the leg. A small incision was made to expose the distal portion of the great (or medial) saphenous vein. A 30 gauge needle catheter was inserted into the distal vein and advanced so that the tip of the needle was positioned just above the knee in an antegrade orientation. A syringe pump was used to inject an efflux enhancer solution (42 μg papaverine in 0.25 ml saline) at a flow rate of 4.5 ml/min followed 1-5 min later by injection of 1.0 ml saline containing 10 μg pDNA per injection at a flow rate of 4.5 ml/min. The solution was injected in the direction of normal blood flow through the vein. Two minutes after injection, the tourniquet was removed and bleeding was controlled with pressure and a hemostatic sponge. The incision was closed with 4-0 Vicryl suture. The procedure was completed in ˜10 min.

As controls, two mice were immunized via plasmid delivery to the liver using tail vein injections (retrograde injection). Mice received injections on the same day as indicated above. For the tail vein injections, 10 μg plasmid DNA in 2.5 ml Ringer's solution per injection was injected into the tail vein using a 27 gauge needle. The entire volume was delivered in less than 10 sec.

To monitor induction of an immune reaction to luciferase, the animals were bled on days 0, 13, 20, 27, 34, 41 and 48. The blood was allowed to clot and the sample was centrifuged to recover the sera. Sera were analyzed for the presence of antibodies to luciferase u sing an ELISA, as follows: 96-well plates were coated with a recombinant luciferase protein (Promega, Madison, Wis.) by incubation of 100 μl 2 μg/ml protein in 0.1 M carbonated buffer per well. Plates were incubated overnight at 4° C., then washed three times with PBS containing 0.05% Tween 20. Wells are blocked with 200 μl PBS+1% non-fat dried milk for 1.5 h at RT and washed three times as above. Mouse sera were diluted in PBS+1% milk. 100 μl diluted sera were added to wells in duplicate and incubated 1.5 h at RT. The plates were washed three times as above. 100 μl anti-mouse polyvalent antibody conjugated to horseradish peroxidase (Sigma, St. Louis, Mo.) diluted 1:20,000 in the PBS+1% milk buffer was added to each well. The plates are washed five times as above. 100 μl tetramethylbenzidine (Sigma) was added to each well and the samples were allowed to develop. The reaction was stopped by addition of 100 μl 1.0 M H₂SO₄ per well and the absorbance was read at 450 nm. A standard curve was generated using a goat anti-luciferase horseradish peroxidase conjugate (Sigma). The results are shown in the table below. The presence of anti-luciferase antibodies in the mouse sera indicates successful induction of an immune response.

Antibody concentration (μg/ml) in mice genetically immunized via injection of plasmid DNA into either tail vein or saphenous vein.

| | | saphenous |
|---|---|---|
| day | tail vein | vein |
| 0 | 0.13 | 0.09 |
| 13 | 0.06 | 2.03 |
| 20 | 1.72 | 51.6 |
| 27 | 47.1 | 175 |
| 34 | 106 | 471 |
| 41 | 174 | 332 |
| 48 | 235 | 393 |

### Example 10

Intravascular genetic immunization via injection into limb vein. Four ˜150 g Sprague-Dawley rats per group were immunized with 500 μg pMIR48. Group 1 animals were immunized by delivery of antigen-encoding polynucleotide via saphenous vein injection Plasmid DNA in 3 ml of normal saline solution (NSS) was used for each injection. Blood flow to and from the limb was restricted just prior to and during the injection, and for 2 min post-injection by placing a tourniquet around the upper leg Oust proximal to/or partially over the quadriceps muscle group). The solution was injected into the great saphenous vein of the distal hind limb at a rate of 3 ml per ˜20 seconds (10 ml/min). The intravenous injections were performed in an anterograde direction (i.e., with the blood flow) via a needle catheter connected to a programmable Harvard PHD 2000 syringe pump (Harvard Instruments). Group 2 animals received immunization via hydrodynamic delivery of polynucleotide through the tail vein. Immunizations occurred on days 0, 13, 20, 20, 27, 25 and 42 and animals were bled on days 7, 20 and 28. Sera were separated and tested in a single ELISA.

Results are shown in μg/ml antibody concentration.

| Day | Tail vein | Saphenous vein |
|---|---|---|
| 0 | 0.15 ± 0.10 | 0.34 ± 0.28 |
| 13 | 0.26 ± 0.11 | 13.3 ± 19.8 |
| 20 | 0.31 ± 0.13 | 62.9 ± 75.3 |
| 27 | 0.42 ± 0.07 | 102 ± 102 |
| 35 | 0.75 ± 0.53 | 469 ± 308 |
| 42 | 0.61 ± 0.27 | 490 ± 370 |

### Example 11

Antibody generation via intravascular genetic immunization works in larger animals as well as mice, as demonstrated in rabbits. Four rabbits were injected on days 0, 14, 21 and 28 with a plasmids encoding the firefly luciferase gene under control of the cytomegalovirus promoter (pMIR48) and the ubiquitin C promoter and a hepatic control region for enhancement of long-term expression (pMIR68). Two animals also received a plasmid encoding murine interleukin 2 under control of the cytomegalovirus promoter (pMIR152).

For each injection, a solution containing the plasmid was inserted into the lumen of the saphenous vein as follows: A latex tourniquet was wrapped around the upper hind limb to block blood flow into and out of the leg and tightened into place with a hemostat. Injections were done into either the great or the small saphenous vein. A 23 gauge catheter was inserted, in antegrade orientation, into the lumen of the vein. A syringe pump was used to inject an efflux enhancer solution (1.0 mg papaverine in 6 ml) at a flow rate of 4-5 ml/min. One to five minutes later a solution containing plasmid DNA was injected through the catheter (1 mg/kg pMIR48 or pMIR68; 2 mg/kg pMIR152 in 18-44 ml saline, 14 ml/kg animal weight.) The solution was injected in 18-30 seconds (1-2 ml/sec). The volume of solution and rate of injection were varied depending on the weight of the rabbit. The solution was injected in the direction of normal blood flow through the vein. The tourniquet was removed two minutes after the injection. Bleeding from the incision and vein puncture was controlled with pressure and a hemostatic sponge. The incision was closed with 4-0 Braunamid suture. The procedure was completed in 20 min.

To monitor induction of an immune reaction to luciferase in the animals, animals were bled via the ear vein. The presence of antibodies in the sera, indicating induction of an immune response, was determined by ELISA and Western blot. The results are shown in FIG. 7. The presence of anti-luciferase antibodies in the rabbit sera indicates successful induction of an immune response. These results demonstrate the applicability of intravascular genetic immunization in larger animals that can be used to produce polyclonal antibodies on a larger scale.

### Example 12

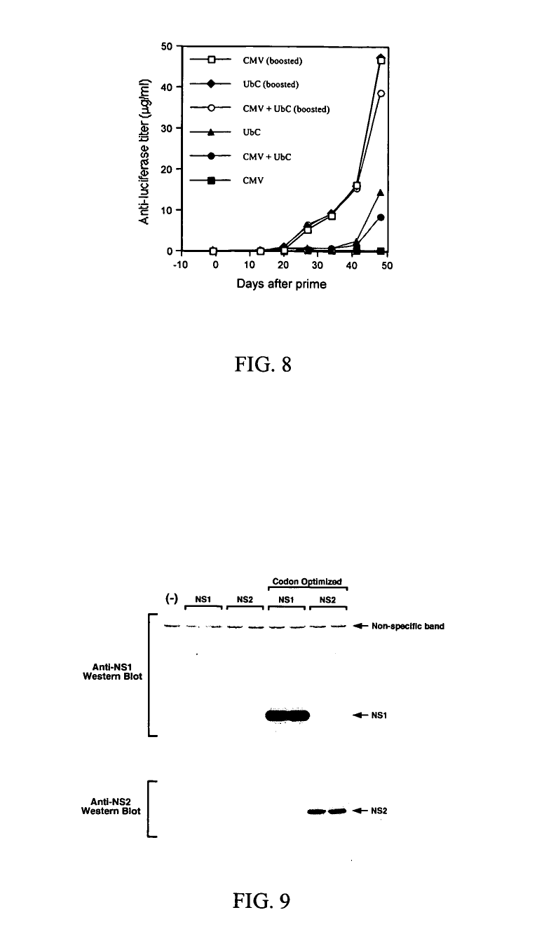

Comparison: Immunogen expression vectors. To test if sustained expression of the antigen may require fewer boosts, we compared genetic immunization of two luciferase vectors: pMIR48 (CMV promoter) and pMIR68 (ubiquitin C promoter). Previous experiments have shown that pMIR68 generates stable luciferase expression for many months at a level about ten-fold below CMV-driven peak levels. FIG. 8 shows the result of antibody induction following hydrodynamic tail vein delivery of 10 μg pMIR48 or pMIR68 alone or a combination of the two (5 μg each). We also compared the effect of 2 boosts versus no boost. A single delivery of pMIR48 did not result in significant antibody titers by day 42, whereas pMIR68 immunized mice did show significant levels of anti-luciferase antibodies. Boosting increased antibody levels significantly, and no differences between the different plasmid DNA groups were observed.

### Example 13

Enhanced immune response by co-delivery of cytokine expression vectors. The immunomodulator Flt3-Ligand (Flt3-L) acts on CD34+progenitor cells and results in increases in DC and NK cells. Intravascular delivery of a CMV promoter-driven Flt3-L vector into ICR mice via tail vein injection was performed to determine the effects of delivery of the Flt3-L gene. Different levels of the expression vector were injected and the number and composition of spleen cells was analyzed after 10 days. Delivery of 10 μg murine Flt3-Ligand pDNA increased the total splenocyte count 3.8 fold (260 million cells per spleen for Flt3-L treated mice compared to 68 million cells per spleen for control mice). Furthermore, the splenocytes demonstrated an increase in the percentage of CD11c+dendritic cells. 2.3% CD11c+splenocytes were observed in control mice while 24.5% CD11c+splenocytes were observed in mice receiving Flt3-L pDNA. A dose-dependent response in total number of splenocytes and CD11c+cells was observed when delivering a range of 1-50 μg/mouse of Flt3-L pDNA.

Examples 14. Codon optimization. Many viruses such as Respiratory Syncytial Virus (RSV) and SARS CoV replicate in the cytoplasm of infected cells and use their own virally encoded polymerases and transcriptases. When genes from such viruses are expressed from mammalian expression cassettes, they are subject to the normal host nuclear processes such as polyadenylation, splicing, and RNA polymerase II mediated transcription. This may lead to incorrect or low levels of expression. Therefore, in order to produce high levels of gene product, it may be important that the sequence encoding that gene be altered to mimic a typical nuclear gene. This codon optimization entails constructing the gene using frequently used codons according to codon-usage tables for the host species and eliminating potential splicing, polyadenylation, and anti-sense start sites present in the native microbial sequence.

To illustrate both the utility and importance of codon optimization, the human RSV mRNAs encoding the non-structural proteins, NS 1 and NS2, were cloned using standard RT-PCR from total cellular RNA made from RSV infected HEp-2 cells. The RSV ORFs were cloned into standard expression vectors downstream of the CMV immediate early promoter. The same RSV NS 1 and NS2 ORFs were also codon optimized and the resulting ORFs were synthesized. These new NS 1 and NS2 encoding DNA fragments were cloned into the same expression vectors. All four expression vectors have identical and optimal translational context surrounding their ATG start codons based on Kozak's rules. Each of the four NS 1 and NS2 expression vectors were then transfected into HeLa cells, and total cell lysates were prepared 24 hours post-transfection for Western blotting. As illustrated in FIG. 9, there was no detectable expression of NS1 and NS2 from the expression vectors containing the non-optimized ORFs. However, transfection of the plasmids containing the optimized ORFs led to high-level expression of both NS 1 and NS2.

The foregoing is considered as illustrative only of the principles of the invention. Furthermore, since numerous modifications and changes will readily occur to those skilled in the art, it is not desired to limit the invention to the exact construction and operation shown and described. Therefore, all suitable modifications and equivalents fall within the scope of the invention.

1. A method for inducing an immune response in a vertebrate against a tumor cell antigen comprising:
a) forming a polynucleotide containing a coding sequence for said antigen operably linked to a promoter;
b) inserting said polynucleotide into a vessel in said vertebrate thereby delivering said polynucleotide to an extravascular cell in said vertebrate; and,
c) expressing said antigen in said cell thereby inducing said immune response.

2. The method of claim 1 wherein said immune response comprises: a cellular immune response.

3. The method of claim 2 wherein said cellular immune response results in T cell mediated killing of tumor cells in said vertebrate.

4. The method of claim 3 wherein said tumor cell consists of a cancer cell.

5. The method of claim 1 wherein said immune response comprises: a humoral immune response.

6. The method of claim 5 wherein said humoral response consists of producing antigen-specific antibodies.

7. A method for genetically immunizing a vertebrate comprising:
a) forming a polynucleotide containing a coding sequence for an antigen operably linked to a promoter;
b) inserting said polynucleotide into a vessel in said vertebrate thereby delivering said polynucleotide to an extravascular cell in said vertebrate; and,
c) expressing said antigen in said cell thereby eliciting an immune response against said antigen.

8. The method of claim 7 wherein said immune response against said antigen cross reacts with a protein associated with an infectious agent.

9. The method of claim 8 wherein genetically immunizing said vertebrate protects said vertebrate of infection by said infectious agent.

10. The method of claim 8 wherein genetically immunizing said vertebrate provides a therapeutic treatment of an infection by said infectious agent.

11. The method of claim 7 wherein said immune response against said antigen cross reacts with a protein associated with a cancer or tumor cell.

12. The method of claim 11 wherein said immune response provides a therapeutic benefit to said vertebrate.

13. The method of claim 7 wherein eliciting an immune response against said antigen comprises: stimulating immune cells of said vertebrate to produce antibodies to said antigen.

14. The method of claim 13 further comprising: collecting antibodies from said vertebrate.

15. The method of claim 13 further comprising: isolating antibody-producing cells from said vertebrate.

16. The method of claim 13 wherein said vertebrate is selected from the list consisting of:
rabbit, mouse, rat, hamster, guinea pig, chicken, donkey, horse, and goat.

17. A method for producing antigen-specific antibodies comprising:
a) forming a polynucleotide containing a coding sequence for an antigen operably linked to a promoter;
b) inserting said polynucleotide into a vessel in a vertebrate thereby delivering said polynucleotide to an extravascular cell in said vertebrate; and,
c) expressing said antigen in said cell thereby eliciting a humoral immune response against said antigen.

18. The method of claim 17 further comprising collecting serum from said vertebrate wherein said serum contains antigen-specific polyclonal antibodies.

19. The method of claim 18 further comprising: isolating antibodies from said serum.

20. The method of claim 19 further comprising: purifying said antigen-specific antibodies.

21. The method of claim 17 further comprising:
a) isolating antibody-producing B lymphocytes from said vertebrate; and,
b) immortalizing said B lymphocytes.

22. The method of claim 21 wherein immortalizing said B lymphocytes comprises:
a) fusing said B lymphocytes with myeloma cells to form antibody-producing hybridoma cells;
b) selecting hybridoma cells that secrete antibodies specific to said antigen; and,
c) clonally growing said selected hybridoma cells.

23. The method of claim 22 wherein said selected hybridoma cells are used to produce monoclonal antibodies.

24. The method of claim 21 wherein immortalizing said B lymphocytes comprises:
retrovirally transducing said B lymphocytes with ABL-Myc.

25. The method of claim 24 wherein said selected transduced B lymhocytes provide a source of monoclonal antibodies.

26. The method of claim 17 wherein said vertebrate is selected from the list consisting of:
rabbit, mouse, rat, hamster, guinea pig, chicken, donkey, horse, and goat.

27. A kit for genetic immunization comprising:
a) a receptacle containing a polynucleotide containing a coding sequence for an antigen operably linked to a promoter,
b) instructions for genetically immunizing a vertebrate with said polynucleotide.
